(19) United States
(12) Patent Application Publication
(10) Pub. No.: US 2024/0382464 A1
(43) Pub. Date: Nov. 21, 2024

(54) Manufacturing Process for Metaxalone Tablets

(71) Applicant: Primus Pharmaceuticals, inc., Scottsdale, AZ (US)

(72) Inventors: Trevor T. Hall, Scottsdale, AZ (US); Ryan E. Hartung, Scottsdale, AZ (US); Accursio B. LaBella, Scottsdale, AZ (US); Sam G. Salamone, Scottsdale, AZ (US); James D. Weir, Scottsdale, AZ (US)

(21) Appl. No.: 18/198,266

(22) Filed: May 16, 2023

Related U.S. Application Data
(60) Provisional application No. 63/344,521, filed on May 20, 2022.

(51) Int. Cl. A61K 31/421; A61K 9/20

(52) U.S. Cl. CPC A61K 31/421; A61K 9/2013; A61K 9/2018; A61K 9/2027; A61K 9/205; A61K 9/2077

(57) ABSTRACT

Methods of making metaxalone tablets having consistent dissolution properties and bioavailability, and tablets manufactured thereby.

### FIELD OF THE INVENTION

The present invention relates to methods of manufacturing metaxalone tablets, and to the tablets produced thereby.

### BACKGROUND OF THE INVENTION

Metaxalone (Skelaxin®), known chemically as 5-[(3,5-dimethylphenoxy) methyl]-2-oxazolidinone, has the following chemical structure:

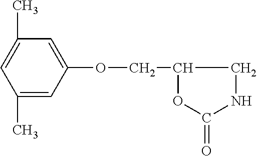 [SMILES: Cc1cc(C)cc(OCC2CNC(=O)O2)c1]

Skelaxin is indicated as an adjunct to rest, physical therapy, and other measures for the relief of discomforts associated with acute, painful musculoskeletal conditions. The mode of action of this drug has not been clearly identified but may be related to its sedative properties. Metaxalone does not directly relax tense skeletal muscles in man. The commercially available tablet contains: metaxalone, 400 and 800 mg, along with inert compression tableting excipients.

Preparation of metaxalone is described in Lunsford et al., J. Am. Chem. Soc. 82, 1166 (1960) and U.S. Pat. No. 3,062,827 to Lunsford (Nov. 6, 1962, Assignee A. H. Robins), which is incorporated herein in its entirety by reference. The '827 patent discloses the compound and related species as anticonvulsants and antispasmodics; however, these activities have not been borne out by clinical experience.

### SUMMARY OF INVENTION

Methods have unexpectedly been developed for the manufacture of metaxalone tablets having consistent dissolution properties and bioavailability. It has also unexpectedly been discovered that, by practicing the methods of the current invention, one is able to produce metaxalone tablets having a stable dissolution profile over time, relative to identical qualitative and quantitative tablets in the prior art not produced using the methods of the current invention. Thus, in a first principal embodiment the invention provides an immediate release metaxalone tablet comprising metaxalone and one or more pharmaceutically acceptable tableting excipients that maintains a stable dissolution profile at accelerated conditions, 40±2° C. and 75%±5% relative humidity, over a period of 6 months or more, when tested at 90 minutes under the dissolution testing described in USP, Metaxalone Tablets, comprising from 500 to 750, from 600 to 680, or 640 mg metaxalone.

In another principal embodiment the invention provides a method of making a first batch of metaxalone tablets comprising: (a) providing a first batch quantity of a first grade of metaxalone or a pharmaceutically acceptable salt thereof (the first FGM quantity); (b) providing a first batch quantity of a second grade of metaxalone or a pharmaceutically acceptable salt thereof (the first SGM quantity); (c) providing a first batch quantity of one or more pharmaceutically acceptable excipients (the first excipient quantity); (d) compressing a plurality of tablet prototypes at a plurality of hardness values ranging from 6 to 35 kp, from 6 to 25 kp, or from 6 to 17 kp, from the first FGM quantity at an FGM proportion, the first SGM quantity at an SGM proportion, and the first excipient quantity at an excipient proportion; (e) providing a first dissolution specification for the prototypes measured according to a first dissolution protocol; (f) first testing the prototypes according to the first dissolution protocol; (g) determining from the first testing a hardness for the prototypes that complies with the first dissolution specification (the first target hardness); and (h) compressing a batch of tablets from the first FGM quantity at the FGM proportion, the first SGM quantity at the SGM proportion, and the first excipient quantity at the excipient proportion, to the first target hardness.

The invention also relates to pluralities tablets produced by the methods of the current invention. In another principal embodiment the invention provides a plurality of compressed metaxalone tablets from first and second batches, wherein: (a) the tablets are characterized by: (i) a proportion of a first grade of metaxalone or a pharmaceutically acceptable salt thereof (the FGM proportion); (ii) a proportion of a second grade of metaxalone or a pharmaceutically acceptable salt thereof (the SGM proportion); (iii) a proportion of one or more pharmaceutically acceptable excipients (the excipient proportion); and (iv) an average hardness value for the plurality; and (b) at least 70%, 80%, or 90% of the tablets from the plurality are characterized by a hardness differing by at least 10%, 20%, 40%, or 60% from the average hardness value.

The invention also relates to methods that ensure a batch of immediate release tablets is bioequivalent in the fed and fasted states, using two dissolution time points to control the release process. Thus, in another principal embodiment the invention provides a method of manufacturing a batch of immediate release metaxalone tablets comprising from 500 to 750, from 600 to 680, or 640 mg metaxalone, bioequivalent in the fed and fasted states, comprising: (a) providing a batch quantity of metaxalone or a pharmaceutically acceptable salt thereof; (b) providing a batch quantity of one or more tableting excipients; (c) compressing the metaxalone and tableting excipients into the plurality of immediate release tablets; and (d) subjecting two or more of the plurality of tablets to a multiple time-point dissolution protocol comprising determining the amount of metaxalone in the tablets released at two or more time points from 30 to 60 minutes apart under dissolution testing described in USP, Metaxalone Tablets (January 2022). In a preferred embodiment, first time point is between 15 and 60 minutes (most preferably 30 minutes), and the second time point is between 60 and 120 minutes (most preferably 90 minutes), endpoints included.

Additional advantages of the invention are set forth in part in the description which follows, and in part will be obvious from the description or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

When particle sizes are based on their percentage difference in size from a defined value, it will be understood that the percentage difference is measured by the following formula when the particles are smaller than the defined value: (size_{defined}−Sizeₛₘₐₗₗ)/(size_{defined}+Sizeₛₘₐₗₗ) times 100%; and the following formula when the particles are larger than the defined value: (size_{large}−size_{defined})/(size_{defined}+size_{large}) times 100%.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 depicts representative distributions of particles sizes from representative batches of first (FGM) and second (SGM) grades of metaxalone used in the manufacturing processes of the current invention.

FIG. 2 depicts dissolution test results from four tablets having different water contents but compressed at the same hardness, as reported in Example 3.

FIG. 3 depicts dissolution test results from tablets produced from the same granulate compressed at a range of hardness values, ranging from 6.2 to 16.3 kp, as reported in Example 4.

FIG. 4 is a graphical plot of dissolution versus hardness for the tablets manufactured according to Example 4.

FIG. 5 depicts the dissolution profile over time of six batches of tablets tested according to the methods described in Example 5.

### DETAILED DESCRIPTION

### Definitions and Use of Terms

When the singular forms “a,” “an” and “the” or like terms are used herein, they will be understood to include plural referents unless the context clearly dictates otherwise. Thus, for example, reference to “an excipient” includes mixtures of two or more such excipients, and the like. The word “or” or like terms as used herein means any one member of a particular list and also includes any combination of members of that list.

When used herein the term “about” or “ca.” will compensate for variability allowed for in the pharmaceutical industry and inherent in pharmaceutical products, such as differences in product strength and bioavailability due to manufacturing variations and time-induced product degradation. The term allows for any variation which in the practice of pharmaceuticals would allow the product being evaluated to be considered pharmaceutically equivalent or bioequivalent, or both if the context requires, to the recited strength of a claimed product. It will be understood that all numeric values expressed in this document can be prefaced by the term “about.”

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality of components, steps or conditions, it will be understood that the element can also be described as comprising any combination of such plurality, or “consisting of” or “consisting essentially of” the plurality or combination of components, steps or conditions.

When ranges are given by specifying the lower end of a range separately from the upper end of the range, or specifying particular numerical values, it will be understood that a separate range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

The term “bioequivalent” has the meaning ascribed to the by the United States Food and Drug Administration in Statistical Approaches to Establishing Bioequivalence, docket number 01D-0027 February 2001.

The term “release” refers to the process of releasing a drug product from a manufacturing facility for human consumption, as defined more specifically at 21 C.F.R. Section 211.165, “Testing and release for distribution.” (January 2022)

As used herein, “therapeutically effective amount” refers to an amount sufficient to elicit the desired biological response. The therapeutically effective amount or dose will depend on the age, sex and weight of the patient, and the current medical condition of the patient. The skilled artisan will be able to determine appropriate dosages depending on these and other factors in addition to the present disclosure.

“Pharmaceutically acceptable” means that which is useful in preparing a pharmaceutical composition that is generally safe, non-toxic and neither biologically nor otherwise undesirable and includes that which is acceptable for human or veterinary pharmaceutical use. “Pharmaceutically acceptable salts” means salts that are pharmaceutically acceptable, as defined above, and which possess the desired pharmacological activity.

When a dose of a drug or its pharmaceutically acceptable salt is described herein, it will be understood that the dose is based on the weight of the free base, excluding any hydrates or solvates thereof, unless the description states that the dose is based on the weight of the salt, hydrate or solvate.

Throughout the patent application, wherever an analysis by a method prescribed in the United States Pharmacopoeia (“USP”) is prescribed, it will be understood that the analysis is performed in accordance with the USP volume in effect on May 15, 2023. It will also be understood that the test need not have been performed, but that the test, if performed, would yield the claimed result. In like manner, any terms not otherwise defined herein can be defined by reference to the USP volume in effect on May 15, 2023.

USP dissolution conditions for metaxalone tablets as that term is used herein means:
Medium: 0.5% sodium lauryl sulfate; 900 mL
Apparatus 2: 100 rpm

The standard USP timepoint for metaxalone tablets is 60 minutes, but it will be understood that the time point will differ according to the methods discussed herein. A 640 mg tablet will, at 30 minutes, have released 26-46% of its metaxalone and, at 90 minutes, will have released no less than 80% of its metaxalone.

A “batch” has the meaning given in FDA's Code of Federal Regulations at 21 CFR 210.31 (January 2022), and is defined as a certain amount of a drug or other material that is intended to have a uniform character and quality within defined limits and is manufactured according to a single production order during the same manufacturing cycle. This definition applies to a batch of metaxalone or a batch of metaxalone tablets.

All testing described herein is conducted in accordance with guidances and requirements published by the United States Food and Drug Administration and the United States Pharmacopoeia, applicable to prescription pharmaceutical products, in effect on Jan. 1, 2022.

### Principal Embodiments

The invention is described herein in terms of principal embodiments and subembodiments. It will be understood that each of the subembodiments can modify any of the principal embodiments, unless such modification is logically inconsistent or expressly disallowed in this document. It will be further understood that the principal embodiments can be combined in any manner, and that the subembodiments can be combined in any manner to further modify any of the principal embodiments, unless such combination is logically inconsistent or expressly disallowed in this document.

In a first principal embodiment the invention provides an immediate release metaxalone tablet comprising metaxalone and one or more pharmaceutically acceptable tableting excipients that maintains a stable dissolution profile, particularly under accelerated conditions over a period of 3 or 6 months or more, when tested at 90 minutes under the dissolution testing described in USP, Metaxalone Tablets, comprising from 500 to 750, from 600 to 680, or 640 mg metaxalone.

In another principal embodiment the invention provides a method of making a first batch of metaxalone tablets comprising: (a) providing a first batch quantity of a first grade of metaxalone or a pharmaceutically acceptable salt thereof (the first FGM quantity); (b) providing a first batch quantity of a second grade of metaxalone or a pharmaceutically acceptable salt thereof (the first SGM quantity); (c) providing a first batch quantity of one or more pharmaceutically acceptable excipients (the first excipient quantity); (d) compressing a plurality of tablet prototypes at a plurality of hardness values ranging from 6 to 35 kp, from 6 to 25 kp, or from 6 to 17 kp, from the first FGM quantity at an FGM proportion, the first SGM quantity at an SGM proportion, and the first excipient quantity at an excipient proportion; (e) providing a first dissolution specification for the prototypes measured according to a first dissolution protocol; (f) first testing the prototypes according to the first dissolution protocol; (g) determining from the first testing a hardness for the prototypes that complies with the first dissolution specification (the first target hardness); and (h) compressing a batch of tablets from the first FGM quantity at the FGM proportion, the first SGM quantity at the SGM proportion, and the first excipient quantity at the excipient proportion, to the first target hardness.

In another principal embodiment the invention provides a plurality of compressed metaxalone tablets from first and second batches, wherein: (a) the tablets are characterized by: (i) a proportion of a first grade of metaxalone or a pharmaceutically acceptable salt thereof (the FGM proportion); (ii) a proportion of a second grade of metaxalone or a pharmaceutically acceptable salt thereof (the SGM proportion); (iii) a proportion of one or more pharmaceutically acceptable excipients (the excipient proportion); and (iv) an average hardness value for the plurality; and (b) at least 70%, 80%, or 90% of the tablets from the plurality are characterized by a hardness differing by at least 10%, 20%, 40%, or 60% from the average hardness value.

In another principal embodiment the invention provides a method of manufacturing a batch of immediate release metaxalone tablets comprising from 500 to 750, from 600 to 680, or 640 mg metaxalone, bioequivalent in the fed and fasted states, comprising: (a) providing a batch quantity of metaxalone or a pharmaceutically acceptable salt thereof; (b) providing a batch quantity of one or more tableting excipients; (c) compressing the metaxalone and tableting excipients into the plurality of immediate release tablets; and (d) subjecting two or more of the plurality of tablets to a multiple time-point dissolution protocol comprising determining the amount of metaxalone in the tablets released at two or more time points separated by from 30 to 60 minutes under dissolution testing described in USP, Metaxalone Tablets (January 2022). In a preferred embodiment, first time point is between 15 and 60 minutes (most preferably 30 minutes), and the second time point is between 60 and 120 minutes (most preferably 90 minutes), endpoints included.

### Subembodiments

The invention can further be defined in terms of various subembodiments, each of which can modify any of the principal embodiments singularly or in any combination.

In a particularly preferred embodiment, (a) the first FGM quantity is characterized by a d50 of from 7 to 86 μm when tested according to USP <429> Laser Light Diffraction; and (b) the first SGM quantity if characterized by a d50 of from 98 to 516 μm when tested according to USP <429> Laser Light Diffraction.

Alternatively, (a) the first FGM quantity can be characterized by a d50 of from 10 to 80 μm when tested according to USP <429> Laser Light Diffraction; and (b) the first SGM quantity can be characterized by a d50 of from 120 to 500 μm when tested according to USP <429> Laser Light Diffraction.

Alternatively, (a) the first FGM quantity can be characterized by a d50 of from 8 to 30 μm when tested according to USP <429> Laser Light Diffraction; and (b) the first SGM quantity can be characterized by a d50 of from 163 to 310 μm when tested according to USP <429> Laser Light Diffraction.

Alternatively or in addition, the first SGM quantity and second SGM quantity can be evaluated in terms of their respective sizes. Thus, in various embodiments, the d50 of the first SGM quantity is at least 2 times, 10 times, 50 times, or 100 times greater than the d50 of the first FGM quantity when tested according to USP <429> Laser Light Diffraction.

The formulations of the present invention can also be defined in terms of metaxalone particle size. In one subembodiment the formulation comprises from 40 to 80 wt % first grade particles of metaxalone and from 20 to 60 wt % second grade particles of metaxalone. In one particular subembodiment the formulation comprises from 30 to 50 wt % or from 35 to 45 wt % first grade particles of metaxalone and from 50 to 70 wt % or from 55 to 65 wt % second grade particles of metaxalone.

The batch sizes manufactured according to the present invention can vary among commercial operations. Thus, in various embodiments, the first batch comprises greater than 10,000, 40,000, 100,000 or 1,000,000 tablets.

Other embodiments are tied to the preferred wet granulation methods of the present invention. Thus, in another embodiment, the methods of the current invention further comprise, prior to compressing step (d), granulating one or more of the pharmaceutical excipients with all or a portion of the FGM proportion and/or SGM proportion, with a percentage of water ranging from 10% to 20%, or from 12% to 15% (w/w).

Other embodiments can be defined based on the ratio of the FGM proportion and the SGM proportion used in the manufacture of the tablets. In one embodiment, the sum of the FGM proportion and the SGM proportion equals from 70 to 98% or from 80 to 90% of the weight of the tablets. In another embodiment the weight ratio of the FGM proportion to the SGM proportion is from 10:1 to 1:10, from 2:1 to 1:2, or from 2:1 to 1:1.

Other embodiments can be defined based on the pharmaceutically acceptable excipients. In one embodiment, the pharmaceutically acceptable excipients are selected from diluents, binders, disintegrants, and lubricants. In another embodiment the excipient proportion equals from 2 to 30% or from 10 to 20% of the weight of the tablets.

In still further embodiments the formulations of the present invention are defined based on the ingredients used to make the formulation. Thus, in one subembodiment, the formulations of the present invention comprise 640 weight parts metaxalone and from 10 to 30 weight parts or from 15 to 25 weight parts propylene glycol alginate.

In still further subembodiments of formulations containing propylene glycol alginate, the formulations comprise from 20 to 35 weight parts or from 24 to 31 weight parts lactose monohydrate; from 10 to 30 or from 15 to 25 weight parts alginic acid; from 40 to 60 weight parts or from 45 to 55 weight parts of povidone; and from 2 to 8 weight parts or from 4 to 6 weight parts of a lubricant. A preferred lubricant is magnesium stearate.

The methods can also be practiced by implementing two distinct dissolution specifications that the tablets must satisfy (at the prototype stage and the batch release stage). Thus, in one embodiment, the method further comprises (a) providing a second dissolution specification for the prototypes measured according to a second dissolution protocol; (b) second testing the prototypes according to the second dissolution protocol; (c) determining from the second testing a hardness for the prototypes that complies with the first dissolution specification and the second dissolution specification (the final target hardness); and (d) compressing the batch of tablets to the final target hardness.

In a preferred embodiment, (a) the first specification is based on a percentage of metaxalone in the prototypes released at 30 minutes; and (b) the second specification is based on a percentage of metaxalone in the prototypes released at 90 minutes.

In a particularly preferred embodiment, (a) the first specification requires from 26 to 46% percent of metaxalone in the prototypes be released at 30 minutes under dissolution testing described in USP, Metaxalone Tablets; and (b) the second specification requires that at least 80 percent of metaxalone in the prototypes be released at 90 minutes under dissolution testing described in USP, Metaxalone Tablets.

The process can be repeated once, twice or any number of times to produce a plurality of batches. Thus, in any of the embodiments of the current invention the method can further comprise making a second batch of tablets from a second batch quantity of the first grade of metaxalone or a pharmaceutically acceptable salt thereof (the second FGM quantity) and/or a second batch quantity of a second grade of metaxalone or a pharmaceutically acceptable salt thereof (the second SGM quantity) by repeating the steps of any of methods of the current invention substituting the second FGM quantity and/or second SGM quantity for the first FGM quantity and/or first SGM quantity, respectively, and keeping all other parameters the same. It will be understood that the second batch need not be in succession to the first batch.

When the method is used to produce multiple batches, the methods can be characterized by additional features. Thus, in one preferred embodiment (a) the first batch of tablets has a first batch hardness value; (b) the first batch hardness value among tablets deviates by no more than 10% or 8%; (c) the second batch of tablets has a second batch hardness value; (d) the second batch hardness value among tablets deviates by no more than 10% or 8%; and (e) the first batch hardness value differs by at least 10%, 20%, 30%, 40% or 60% from the average of the first batch hardness value and the second batch hardness value.

Still further embodiments relate to the plurality of tablets made by any of the methods of the present invention.

Other embodiments relate to the plurality of batches made by any of the methods of the current invention. As mentioned above, one can look at any plurality of batches, not necessarily produced in sequence, to determine whether the invention is practiced.

The plurality of tablets of the current invention, can be characterized by various features. In one embodiment, at least 45% of the tablets are characterized by a hardness substantially equal to a designated percentage greater than the average hardness value and at least 45% of the tablets are characterized by a hardness substantially equal to the designated percentage less than the average hardness value, wherein the designated percentage is at least 10%, 20%, 40%, or 60%.

In another embodiment of the plurality of tablets, obtained from a plurality of batches, (a) the first batch of tablets has a first batch hardness value; (b) the first batch hardness value among tablets deviates by no more than 10% or 8%; (c) the second batch of tablets has a second batch hardness value; (d) the second batch hardness value among tablets deviates by no more than 10% or 8%; and (e) the first batch hardness value differs by at least 10%, 20%, 30%, 40% or 60% from the average of the first batch hardness value and the second batch hardness value.

In one particular embodiment, the sum of the FGM proportion and the SGM proportion in the tablets equals from 70 to 98% or from 80 to 90% of the weight of the tablets.

In another particular embodiment, the weight ratio of the FGM proportion to the SGM proportion in the tablets is from 10:1 to 1:10, from 2:1 to 1:2, or from 2:1 to 1:1.

In another particular embodiment, the pharmaceutically acceptable excipients in the tablets are selected from diluents, binders, disintegrants, and lubricants.

In still another particular embodiment, the excipient proportion in the tablets equals from 2 to 30% or from 10 to 20% of the weight of the tablets.

In still another embodiment. the mean particle size of the first FGM in the tablets is from 1 to 60 microns and the mean particle size of the first SGM is from 100 to 1000 microns when tested according to USP <429> Laser Light Diffraction.

In yet another embodiment, (a) the first FGM quantity is characterized by a d50 of from 7 to 86 μm when tested according to USP <429> Laser Light Diffraction; and (b) the first SGM quantity if characterized by a d50 of from 98 to 516 μm when tested according to USP <429> Laser Light Diffraction.

In still another embodiment, (a) the tablets satisfy first and second dissolution specifications; (b) the first specification requires from 26 to 46% percent of metaxalone in the prototypes be released at 30 minutes under dissolution testing described in USP, Metaxalone Tablets; and (c) the second specification requires that at least 80 percent of metaxalone in the prototypes be released at 90 minutes under dissolution testing described in USP, Metaxalone Tablets.

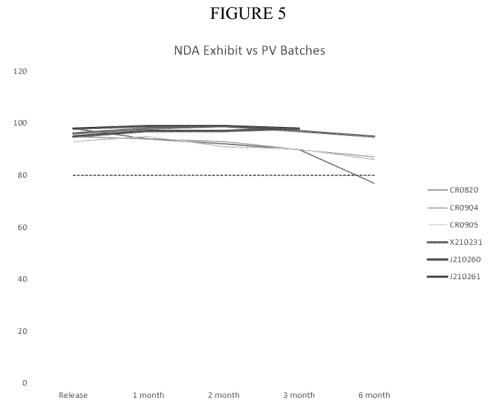

Still other embodiments relate to tablets capable of achieving the stable dissolution profile depicted in FIG. 5. As described herein, this stable dissolution profile is achieved by properly balancing the hardness of the tablet with the particle size of the metaxalone. Thus, in one embodiment the tablets of the current invention comprise means for maintaining a stable dissolution profile, wherein the means corresponds to a balance of hardness and API particle size capable of maintaining the dissolution profile when tested according to the methods described in Example 5 or USP [Metaxalone Tablets]. In a particularly preferred embodiment, the percentage of metaxalone released after 6 month differs by no more than 5%, 3%, or 2%, on an absolute basis, from the percentage of metaxalone released after 0 months or 3 months, when stored at 40±2° C. and 75%±5% relative humidity, preferably at 30 and 90 minute time points.

In another embodiment, the percentage of metaxalone released after 3 month differs by no more than 2%, on an absolute basis, from the percentage of metaxalone released after 0 months, when stored at 40±2° C. and 75%±5% relative humidity, preferably at 30 and 90 minute timepoints when tested according to USP [Metaxalone Tablets].

In still another embodiment, the percentage of metaxalone released after 6 month differs by no more than 3% or 2%, on an absolute basis, from the percentage of metaxalone released after 3 months, when stored at 40±2° C. and 75%±5% relative humidity, preferably at 30 and 60 minute timepoints when tested according to USP [Metaxalone Tablets].

The “absolute basis” of different percentages is determined by subtracting the percent released at one time point from the percent released at another time point. It will be understood that the dissolution testing is performed in accordance with ICH guidelines governing product release, based on a suitable number of tablets, as described in ICH Q6A specifications: test procedures and acceptance criteria for new drug substances and new drug products: chemical substances.

In still further embodiments, the tablets are bioequivalent to Skelaxin® Tablets 800 mg under fasted conditions. In even further embodiments, the tablets are bioequivalent to Skelaxin® Tablets 800 mg under fasted conditions, and the tablets are bioequivalent in the fed and fasted states. Bioequivalence testing is according to FDA's Guidance Document, Bioavailability and Bioequivalence Studies Submitted in NDAs or INDs—General Considerations.

Other embodiments relate to the packaging for the bottles, and the distinct packaging used for tablets from separate batches. Thus, any of the pluralities of tablets of the current invention can be packaged in a plurality of bottles, wherein the tablets from the first batch and the second batch are packaged in separate bottles.

### ADDITIONAL EMBODIMENTS

Additional embodiments are defined as follows:
[Embodiment A] A plurality of immediate release metaxalone tablet of metaxalone and one or more pharmaceutically acceptable tableting excipients that maintains a stable dissolution profile over a period of 6 months or more, when tested at 90 minutes under the dissolution testing described in USP, Metaxalone Tablets, comprising from 500 to 750, from 500 to 680, or 640 mg of metaxalone.
[Embodiment B] The plurality of tablets of embodiment 1, wherein the percentage of metaxalone released after 6 month differs by no more than 5%, 3%, or 2%, on an absolute basis, from the percentage of metaxalone released after 0 months or 3 months, when stored at accelerated condition of 40° C. and 75% relative humidity.
[Embodiment C] The plurality of tablets of embodiment 1 or 2, wherein the hardness of the tablet is balanced with the particle size of the metaxalone to produce the stable dissolution profile.
[Embodiment D] The plurality of tablets of embodiment 1 or 2, comprising means for maintaining a stable dissolution profile.
[Embodiment E] The plurality of tablets of any of embodiments 1-4, made by a process of comprising:
   a) providing a first batch quantity of a first grade of metaxalone or a pharmaceutically acceptable salt thereof (the first FGM quantity);
   b) providing a first batch quantity of a second grade of metaxalone or a pharmaceutically acceptable salt thereof (the first SGM quantity);
   c) providing a first batch quantity of one or more pharmaceutically acceptable excipients (the first excipient quantity);
   d) compressing a plurality of tablet prototypes at a plurality of hardnesses ranging from 6 to 35 kp, from 6 to 25 kp, or from 6 to 17 kp, from the first FGM quantity at an FGM proportion, the first SGM quantity at an SGM proportion, and the first excipient quantity at an excipient proportion;
   e) providing a first dissolution specification for the prototypes measured according to a first dissolution protocol;
   f) first testing the prototypes according to the first dissolution protocol;
   g) determining from the first testing a hardness for the prototypes that complies with the first dissolution specification (the first target hardness); and
   h) compressing a batch of tablets from the first FGM quantity at the FGM proportion, the first SGM quantity at the SGM proportion, and the first excipient quantity at the excipient proportion, to the first target hardness.
[Embodiment F] The plurality of metaxalone tablets of any of the foregoing embodiments, wherein:
   a) the tablets are characterized by:
      i) a proportion of a first grade of metaxalone or a pharmaceutically acceptable salt thereof (the FGM proportion);
      ii) a proportion of a second grade of metaxalone or a pharmaceutically acceptable salt thereof (the SGM proportion);
      iii) a proportion of one or more pharmaceutically acceptable excipients (the excipient proportion); and
      iv) an average hardness value for the plurality; and
   b) at least 70%, 80%, or 90% of the tablets from the plurality are characterized by a hardness differing by at least 10%, 20%, 40%, or 60% from the average hardness value.
[Embodiment G] The plurality of metaxalone tablets of any of the foregoing embodiments bioequivalent in the fed and fasted states, made by a process comprising:
   a) providing a batch quantity of metaxalone or a pharmaceutically acceptable salt thereof;
   b) providing a batch quantity of one or more tableting excipients;
   c) compressing the metaxalone and tableting excipients into the plurality of immediate release tablets; and
   d) subjecting two or more of the plurality of tablets to a multiple time-point dissolution protocol comprising determining the amount of metaxalone in the tablets released at two or more time points from 30 to 60 minutes apart under dissolution testing described in USP, Metaxalone Tablets (January 2022).
[Embodiment H] A method of making a first batch of metaxalone tablets comprising:
   a) providing a first batch quantity of a first grade of metaxalone or a pharmaceutically acceptable salt thereof (the first FGM quantity);
   b) providing a first batch quantity of a second grade of metaxalone or a pharmaceutically acceptable salt thereof (the first SGM quantity);
   c) providing a first batch quantity of one or more pharmaceutically acceptable excipients (the first excipient quantity);
   d) compressing a plurality of tablet prototypes at a plurality of hardnesses ranging from 6 to 35 kp, from 6 to 25 kp, or from 6 to 17 kp, from the first FGM quantity at an FGM proportion, the first SGM quantity at an SGM proportion, and the first excipient quantity at an excipient proportion;
   e) providing a first dissolution specification for the prototypes measured according to a first dissolution protocol;
   f) first testing the prototypes according to the first dissolution protocol;
   g) determining from the first testing a hardness for the prototypes that complies with the first dissolution specification (the first target hardness); and
   h) compressing a batch of tablets from the first FGM quantity at the FGM proportion, the first SGM quantity at the SGM proportion, and the first excipient quantity at the excipient proportion, to the first target hardness.
[Embodiment I] The method of embodiment 8, wherein:
   a) the first FGM quantity is characterized by a d50 of from 7 to 86 μm when tested according to USP <429> Laser Light Diffraction; and
   b) the first SGM quantity if characterized by a d50 of from 98 to 516 μm when tested according to USP <429> Laser Light Diffraction.
[Embodiment J] The method of embodiment 8 or 9, wherein the d50 of the first SGM quantity is at least 2 times, 10 times, 50 times, or 100 times greater than the d50 of the first FGM quantity when tested according to USP <429> Laser Light Diffraction.
[Embodiment K] The method of embodiment 8, wherein the first batch comprises greater than 10,000, 40,000, 100,000 or 1,000,000 tablets.
[Embodiment L] The method of embodiment 8, further comprising, prior to compressing step (d), granulating one or more of the pharmaceutical excipients with all or a portion of the FGM proportion and/or SGM proportion, with a percentage of water ranging from 10% to 20%, or from 12% to 15% (w/w).
[Embodiment M] The method of embodiment 8, wherein the sum of the FGM proportion and the SGM proportion equals from 70 to 98% or from 80 to 90% of the weight of the tablets.
[Embodiment N] The method of embodiment 8, wherein the weight ratio of the FGM proportion to the SGM proportion is from 10:1 to 1:10, from 2:1 to 1:2, or from 2:1 to 1:1.
[Embodiment O] The method of embodiment 8, wherein the pharmaceutically acceptable excipients are selected from diluents, binders, disintegrants, and lubricants.
[Embodiment P] The method of embodiment 8, wherein the excipient proportion equals from 2 to 30% or from 10 to 20% of the weight of the tablets.
[Embodiment Q] The method of embodiment 8, further comprising:
   a) providing a second dissolution specification for the prototypes measured according to a second dissolution protocol;
   b) second testing the prototypes according to the second dissolution protocol;
   c) determining from the second testing a hardness for the prototypes that complies with the first dissolution specification and the second dissolution specification (the final target hardness); and
   d) compressing the batch of tablets to the final target hardness.
[Embodiment R] The method of embodiment 17, wherein:
   a) the first specification is based on a percentage of metaxalone in the prototypes released at 30 minutes; and
   b) the second specification is based on a percentage of metaxalone in the prototypes released at 90 minutes.
[Embodiment S] The method of embodiment 18, wherein:
   a) the first specification requires from 26 to 46% percent of metaxalone in the prototypes be released at 30 minutes under dissolution testing described in USP, Metaxalone Tablets; and
   b) the second specification requires that at least 80 percent of metaxalone in the prototypes be released at 90 minutes under dissolution testing described in USP, Metaxalone Tablets.
[Embodiment T] The method of any of embodiments 8-19, further comprising making a second batch of tablets from a second batch quantity of the first grade of metaxalone or a pharmaceutically acceptable salt thereof (the second FGM quantity) and/or a second batch quantity of a second grade of metaxalone or a pharmaceutically acceptable salt thereof (the second SGM quantity) by repeating the steps of any of embodiments 1-12 substituting the second FGM quantity and/or second SGM quantity for the first FGM quantity and/or first SGM quantity, respectively, and keeping all other parameters the same.
[Embodiment U] The method of embodiment 20, wherein:
   a) the first batch of tablets has a first batch hardness value;
   b) the first batch hardness value among tablets deviates by no more than 10% or 8%;
   c) the second batch of tablets has a second batch hardness value;
   d) the second batch hardness value among tablets deviates by no more than 10% or 8%; and
   e) the first batch hardness value differs by at least 10%, 20%, 30%, 40% or 60% from the average of the first batch hardness value and the second batch hardness value.
[Embodiment V] A plurality of metaxalone tablets produced by the method of any of embodiments 8-21.
[Embodiment W] A plurality of metaxalone batches produced by the method of embodiment 21.
[Embodiment X] A plurality of compressed metaxalone tablets from first and second batches, wherein:
   a) the tablets are characterized by:
      i) a proportion of a first grade of metaxalone or a pharmaceutically acceptable salt thereof (the FGM proportion);
      ii) a proportion of a second grade of metaxalone or a pharmaceutically acceptable salt thereof (the SGM proportion);
      iii) a proportion of one or more pharmaceutically acceptable excipients (the excipient proportion); and
      iv) an average hardness value for the plurality; and
   b) at least 70%, 80%, or 90% of the tablets from the plurality are characterized by a hardness differing by at least 10%, 20%, 40%, or 60% from the average hardness value.
[Embodiment Y] The plurality of tablets of embodiment 24, wherein at least 45% of the tablets are characterized by a hardness substantially equal to a designated percentage greater than the average hardness value and at least 45% of the tablets are characterized by a hardness substantially equal to the designated percentage less than the average hardness value, wherein the designated percentage is at least 10%, 20%, 40%, or 60%.
[Embodiment Z] The plurality of tablets of embodiment 25, wherein:
   a) the first batch of tablets has a first batch hardness value;
   b) the first batch hardness value among tablets deviates by no more than 10% or 8%;
   c) the second batch of tablets has a second batch hardness value;
   d) the second batch hardness value among tablets deviates by no more than 10% or 8%; and
   e) the first batch hardness value differs by at least 10%, 20%, 30%, 40% or 60% from the average of the first batch hardness value and the second batch hardness value.
[Embodiment AA] The plurality of tablets of embodiment 26, wherein the sum of the FGM proportion and the SGM proportion equals from 70 to 98% or from 80 to 90% of the weight of the tablets.
[Embodiment BB] The plurality of tablets of embodiment 26, wherein the weight ratio of the FGM proportion to the SGM proportion is from 10:1 to 1:10, from 2:1 to 1:2, or from 2:1 to 1:1.
[Embodiment CC] The plurality of tablets of embodiment 26, wherein the pharmaceutically acceptable excipients are selected from diluents, binders, disintegrants, and lubricants.
[Embodiment DD] The plurality of tablets of embodiment 26, wherein excipient proportion equals from 2 to 30% or from 10 to 20% of the weight of the tablets.
[Embodiment EE] The plurality of tablets of embodiment 26, wherein the mean particle size of the first FGM is from 1 to 60 microns and the mean particle size of the first SGM is from 100 to 1000 microns when tested according to USP <429> Laser Light Diffraction.
[Embodiment FF] The plurality of tablets of embodiment 26, wherein
   a) the first FGM quantity is characterized by a d50 of from 7 to 86 μm when tested according to USP <429> Laser Light Diffraction; and
   b) the first SGM quantity if characterized by a d50 of from 98 to 516 μm when tested according to USP <429> Laser Light Diffraction.
[Embodiment GG] The plurality of tablets of embodiment 26, wherein:
   a) the tablets satisfy first and second dissolution specifications;
   b) the first specification requires from 26 to 46% percent of metaxalone in the prototypes be released at 30 minutes under dissolution testing described in USP, Metaxalone Tablets; and
   c) the second specification requires that at least 80 percent of metaxalone in the prototypes be released at 90 minutes under dissolution testing described in USP, Metaxalone Tablets.
[Embodiment HH] The plurality of tablets of any of embodiments 26-33, packaged in a plurality of bottles, wherein the tablets from the first batch and the second batch are packaged in separate bottles.
[Embodiment II] A method of manufacturing a batch of immediate release metaxalone tablets bioequivalent in the fed and fasted states, comprising from 500 to 750, from 600 to 680, or 640 mg of metaxalone, comprising:
   a) providing a batch quantity of metaxalone or a pharmaceutically acceptable salt thereof;
   b) providing a batch quantity of one or more tableting excipients;
   c) compressing the metaxalone and tableting excipients into the plurality of immediate release tablets; and
   d) subjecting two or more of the plurality of tablets to a multiple time-point dissolution protocol comprising determining the amount of metaxalone in the tablets released at two or more time points from 30 to 60 minutes apart under dissolution testing described in USP, Metaxalone Tablets (January 2022).
[Embodiment JJ] The method of embodiment 35, wherein the first time point is between 15 and 59 minutes, and the second time point is between 60 and 120 minutes, endpoints included.
[Embodiment KK] The method of embodiment 35, further comprising releasing the batch of tablets if:
   a) from 26 to 46% percent of metaxalone from the two or more tablets is released at 30 minutes under the dissolution testing described in USP, Metaxalone Tablets; and
   b) at least 80% of the metaxalone from the two or more tablets is released at 90 minutes under the dissolution testing described in USP, Metaxalone Tablets.
[Embodiment LL] The method of embodiment 35, wherein the metaxalone equals from 70 to 98% or from 80 to 90% of the weight of the tablets, preferably greater than 82.5% of the weight of the tablets.
[Embodiment MM] The method of embodiment 35, wherein the pharmaceutically acceptable excipients are selected from diluents, binders, disintegrants, and lubricants.
[Embodiment NN] The method of embodiment 35, wherein excipient proportion equals from 2 to 30% or from 10 to 20% of the weight of the tablets, preferably less than 17.5% of the weight of the tablets.
[Embodiment OO] The method of embodiment 35, wherein the batch quantity of metaxalone comprises a first SGM quantity of metaxalone and a first FGM quantity of metaxalone, wherein the d50 of the first SGM quantity is at least 2 times, 10 times, 50 times, or 100 times greater than the d50 of the first FGM quantity when tested according to USP <429> Laser Light Diffraction.
[Embodiment PP] The method of embodiment 41, wherein the weight ratio of the FGM proportion to the SGM proportion is from 10:1 to 1:10, from 2:1 to 1:2, or from 2:1 to 1:1.
[Embodiment QQ] The method of embodiment 41, wherein the mean particle size of the first FGM is from 1 to 60 microns and the mean particle size of the first SGM is from 100 to 1000 microns when tested according to USP <429> Laser Light Diffraction.
[Embodiment RR] The method of embodiment 41, wherein
   a) the first FGM quantity is characterized by a d50 of from 7 to 86 μm when tested according to USP <429> Laser Light Diffraction; and
   b) the first SGM quantity if characterized by a d50 of from 98 to 516 μm when tested according to USP <429> Laser Light Diffraction.
[Embodiment SS] A tablet manufactured by the method of any of embodiments 35-44.
[Embodiment TT] A plurality of batches manufactured by the method of any of embodiments 35-44.
[Embodiment UU] An immediate release metaxalone tablet of metaxalone and one or more pharmaceutically acceptable tableting excipients that maintains a stable dissolution profile over a period of 6 months or more, when tested at 90 minutes under the dissolution testing described in USP, Metaxalone Tablets, comprising from 500 to 750, from 500 to 680, or 640 mg of metaxalone.
[Embodiment VV] The tablet of embodiment 47, wherein the percentage of metaxalone released after 6 month differs by no more than 5%, 3%, or 2%, on an absolute basis, from the percentage of metaxalone released after 0 months or 3 months, when stored at accelerated condition of 40° C. and 75% relative humidity.
[Embodiment WW] The tablet of embodiment 47 or 48, wherein the hardness of the tablet is balanced with the particle size of the metaxalone to produce the stable dissolution profile.
[Embodiment XX] The tablet of embodiment 47 or 48, comprising means for maintaining a stable dissolution profile.
[Embodiment YY] The tablet of any of embodiments 47-50, made by the process of 8-21 or 35-44.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1 Representative Formulation

Table 1 describes a representative batch formulation for a 640 mg tablet of the current invention:

TABLE 1

| | Quantity/ | |
|---|---|---|
| | Batch | |
| Ingredient | (kg) | |
| Metaxalone First Grade (FGM) | 53.760 | kg |
| Metaxalone Second Grade (SGM) | 35.840 | kg |
| Lactose Monohydrate | 3.764 | kg |
| FD&C Yellow #6 | 0.045 | kg |
| Propylene Glycol Alginate | 2.240 | kg |
| Alginic Acid | 2.240 | kg |
| Povidone | 6.720 | kg |
| Purified Water* | 22.8 | kg |
| Magnesium Stearate | 0.672 | kg |
| Total | 105.28 | kg |

*Not present in final product.

Distributions of particles sizes from representative batches of first grade and regular metaxalone are provided in FIG. 1.

### Example 2 Representative Manufacturing Method

This example includes detailed information describing the manner in which Metaxalone Tablets 640 mg are manufactured, using the formulation and metaxalone described in Table 1.
Granulating Solution: Povidone was dissolved by adding to Purified Water while mixing using mixer at required speed.
Pre-Mixing: Metaxalone First Grade, Metaxalone Second Grade, FD&C Yellow #6, Propylene Glycol Alginate, and Alginic Acid were mixed at a suitable head speed.
Wet-Granulation: To the above Pre-Mix blend, granulating solution was added while mixing at suitable head speed.
Drying: Wet-Granulation was dried in an oven dryer to achieve desired moisture content. Milling: Upon completion of drying process, dried granulation was milled using comminuting mill.
Final-Mixing: Magnesium Stearate was added to milled blend and lubricated using suitable blender.
Compression: Final-Mix Blend was compressed into tablets using Rotary Tablet Press.

### Example 3 Determination of Optimum Water for Granulation

Metaxalone tablets having the formulation described in Table 1 were prepared according to a standard wet granulation technique described in Example 2. To reduce the water content, povidone was added dry with the other pre-mix ingredients, and only water was added as the granulating solution. This simplified the process and allowed for a step-wise addition of water to determine optimal water quantity. Samples of the granulation were taken at different water addition levels and evaluated. For the batch, 14.22% water (water/water+solids) was added to produce a fine granular product. The batch was compressed on a Rotary Tablet Press to generate tablets at various hardness values, and submitted for dissolution testing after drying to remove excess water.

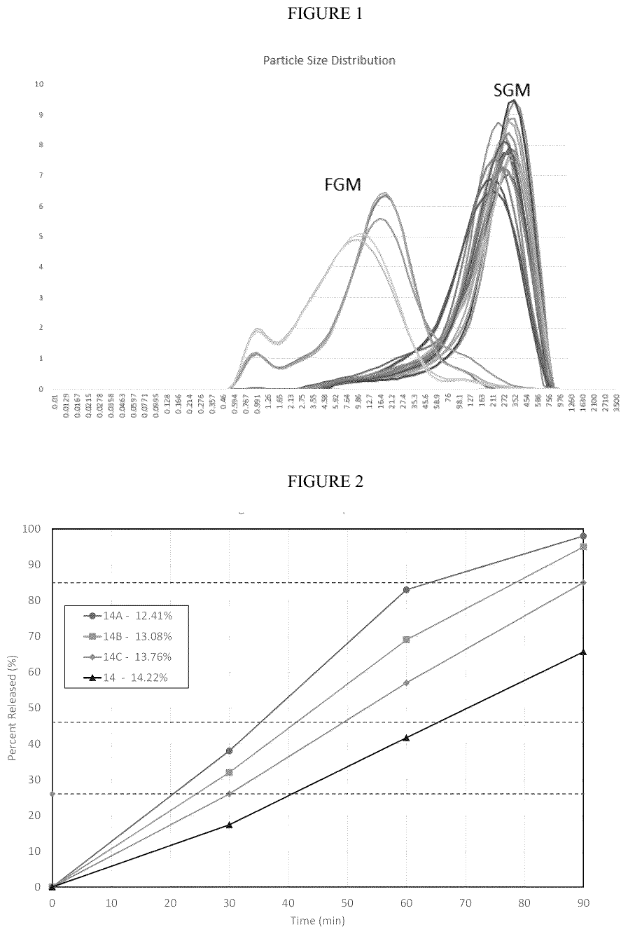

Three samples taken during the granulation step contained less water. These were processed similarly to the main batch and compressed on a tablet press. The batch numbers were designated 14A, 14B, and 14C. Sample 14A contained 12.41% water; sample 14B contained 13.08% water; and sample 14C contained 13.76% water. The final water level of the finished batch contained 14.22% water. The samples were compressed at hardness target and upper and lower hardness values 7.1, 10.7, and 14.3 kp. The upper hardness value samples were tested for dissolution, as reported in FIG. 2.

### Example 4 Determination of Optimum Compression Force

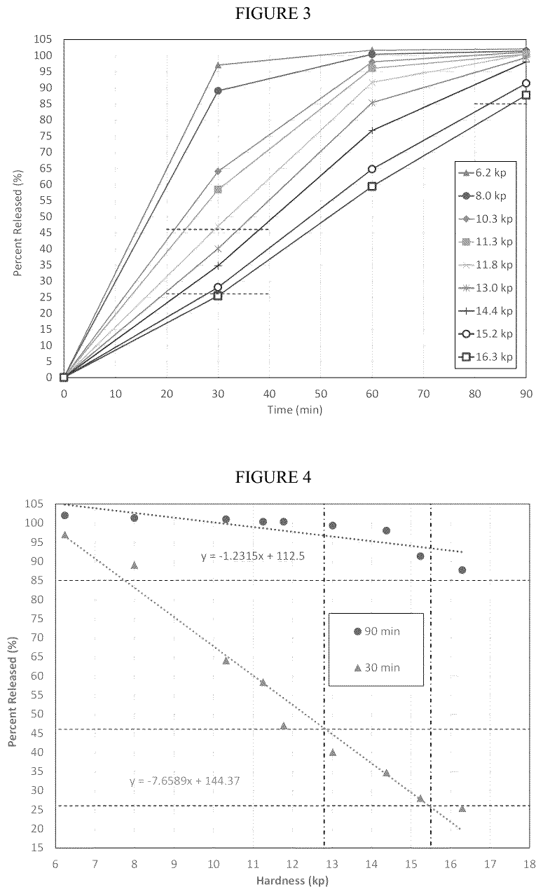

Dissolution was subsequently tested for a single granulate compressed at a range of hardness values, ranging from 6.2 to 16.3 kp. All samples were tested according to USP Metaxalone Tablets monograph and the dissolution is shown in FIG. 3. Dissolution versus hardness is plotted in FIG. 4. The hardness limits were obtained by this method with the minimum value set for the upper hardness limit comparing the 30 and 90 minute trendlines, 12.5 and 15.2 kp. The hardness target was set as the midpoint of the upper and lower specification, 13.9 kp, and the action limits were set at +/−1.0 kp from the target, 12.9 and 14.9 kp. The remainder of the batch was then compressed on a Rotary Tablet Press, at the target hardness.

### Example 5 Release Profile Stability of Tablets

In order to determine the improvement provided by the manufacturing processes disclosed herein, stability testing was performed to determine the stability of the dissolution profile of tablets manufacturing according to the methods described herein, compared to the tablets produced by the methods described in PCT/US2020/039041, each having an identical qualitative/quantitative formulation. In particular, tablets from three batches of product manufactured according to PCT/US2020/039041 (“NDA exhibit” batches) and tablets from three batches manufactured according to the claimed invention (“process validation” or “PV” batches), were stored in a stability chamber at ICH Q1A (R2) accelerated conditions (40±2° C./75%±5% relative humidity) to simulate product shelf life and tested periodically for dissolution kinetics according to USP <711> Dissolution method for the USP Metaxalone Tablets monograph Test 3 acceptance criteria, at 30 minute (PV Batch only) and 90 minute (PV and NDA Exhibit Batches) time points. 90-minute dissolution results are depicted in FIG. 5 and tabulated in Table 2:

TABLE 2
% Dissolution

| | Product | | | | |
|---|---|---|---|---|---|
| | Release | 1 month | 2 month | 3 month | 6 month |
| NDA Exhibit | | | | | |
| CR0820 | 98 | 94 | 92 | 90 | 77 |
| CR0904 | 95 | 94 | 93 | 90 | 87 |
| CR0905 | 93 | 95 | 91 | 90 | 86 |
| PV Batches | | | | | |
| X210231 | 96 | 98 | 99 | 97 | 95 |
| J210260 | 98 | 99 | 99 | 98 | Pending |
| J210261 | 95 | 97 | 97 | 98 | Pending |

A can be seen, the three NDA exhibit batches did not maintain their dissolution stability over time. This is in stark contrast to the three PV batches, which did.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A plurality of immediate release metaxalone tablet of metaxalone and one or more pharmaceutically acceptable tableting excipients that maintains a stable dissolution profile over a period of 6 months or more, when tested at 90 minutes under the dissolution testing described in USP, Metaxalone Tablets, comprising from 500 to 750, from 500 to 680, or 640 mg of metaxalone.

2) The plurality of tablets of claim 1, wherein the percentage of metaxalone released after 6 month differs by no more than 5%, 3%, or 2%, on an absolute basis, from the percentage of metaxalone released after 0 months or 3 months, when stored at accelerated condition of 40° C. and 75% relative humidity.

3) The plurality of tablets of claim 1, wherein the hardness of the tablet is balanced with the particle size of the metaxalone to produce the stable dissolution profile.

4) The plurality of tablets of claim 1, comprising means for maintaining a stable dissolution profile.

5) The plurality of tablets of claim 1, made by a process of comprising:
a) providing a first batch quantity of a first grade of metaxalone or a pharmaceutically acceptable salt thereof (the first FGM quantity);
b) providing a first batch quantity of a second grade of metaxalone or a pharmaceutically acceptable salt thereof (the first SGM quantity);
c) providing a first batch quantity of one or more pharmaceutically acceptable excipients (the first excipient quantity);
d) compressing a plurality of tablet prototypes at a plurality of hardnesses ranging from 6 to 35 kp, from 6 to 25 kp, or from 6 to 17 kp, from the first FGM quantity at an FGM proportion, the first SGM quantity at an SGM proportion, and the first excipient quantity at an excipient proportion;
e) providing a first dissolution specification for the prototypes measured according to a first dissolution protocol;
f) first testing the prototypes according to the first dissolution protocol;
g) determining from the first testing a hardness for the prototypes that complies with the first dissolution specification (the first target hardness); and
h) compressing a batch of tablets from the first FGM quantity at the FGM proportion, the first SGM quantity at the SGM proportion, and the first excipient quantity at the excipient proportion, to the first target hardness.

6) The plurality of metaxalone tablets of claim 1, wherein:
a) the tablets are characterized by:
i) a proportion of a first grade of metaxalone or a pharmaceutically acceptable salt thereof (the FGM proportion);
ii) a proportion of a second grade of metaxalone or a pharmaceutically acceptable salt thereof (the SGM proportion);
iii) a proportion of one or more pharmaceutically acceptable excipients (the excipient proportion); and
iv) an average hardness value for the plurality; and
b) at least 70%, 80%, or 90% of the tablets from the plurality are characterized by a hardness differing by at least 10%, 20%, 40%, or 60% from the average hardness value.

7) The plurality of metaxalone tablets of claim 1 bioequivalent in the fed and fasted states, made by a process comprising:
a) providing a batch quantity of metaxalone or a pharmaceutically acceptable salt thereof;
b) providing a batch quantity of one or more tableting excipients;
c) compressing the metaxalone and tableting excipients into the plurality of immediate release tablets; and
d) subjecting two or more of the plurality of tablets to a multiple time-point dissolution protocol comprising determining the amount of metaxalone in the tablets released at two or more time points from 30 to 60 minutes apart under dissolution testing described in USP, Metaxalone Tablets (January 2022).

8-23) (canceled)

24) A plurality of compressed metaxalone tablets from first and second batches, wherein:
a) the tablets are characterized by:
i) a proportion of a first grade of metaxalone or a pharmaceutically acceptable salt thereof (the FGM proportion);
ii) a proportion of a second grade of metaxalone or a pharmaceutically acceptable salt thereof (the SGM proportion);
iii) a proportion of one or more pharmaceutically acceptable excipients (the excipient proportion); and
iv) an average hardness value for the plurality; and
b) at least 70%, 80%, or 90% of the tablets from the plurality are characterized by a hardness differing by at least 10%, 20%, 40%, or 60% from the average hardness value.

25) The plurality of tablets of claim 24, wherein at least 45% of the tablets are characterized by a hardness substantially equal to a designated percentage greater than the average hardness value and at least 45% of the tablets are characterized by a hardness substantially equal to the designated percentage less than the average hardness value, wherein the designated percentage is at least 10%, 20%, 40%, or 60%.

26) The plurality of tablets of claim 25, wherein:
a) the first batch of tablets has a first batch hardness value;
b) the first batch hardness value among tablets deviates by no more than 10% or 8%;
c) the second batch of tablets has a second batch hardness value;
d) the second batch hardness value among tablets deviates by no more than 10% or 8%; and
e) the first batch hardness value differs by at least 10%, 20%, 30%, 40% or 60% from the average of the first batch hardness value and the second batch hardness value.

27) The plurality of tablets of claim 26, wherein the sum of the FGM proportion and the SGM proportion equals from 70 to 98% or from 80 to 90% of the weight of the tablets.

28) The plurality of tablets of claim 26, wherein the weight ratio of the FGM proportion to the SGM proportion is from 10:1 to 1:10, from 2:1 to 1:2, or from 2:1 to 1:1.

29) (canceled)

30) The plurality of tablets of claim 26, wherein excipient proportion equals from 2 to 30% or from 10 to 20% of the weight of the tablets.

31) The plurality of tablets of claim 26, wherein the mean particle size of the first FGM is from 1 to 60 microns and the mean particle size of the first SGM is from 100 to 1000 microns when tested according to USP <429> Laser Light Diffraction.

32) The plurality of tablets of claim 26, wherein
a) the first FGM quantity is characterized by a d50 of from 7 to 86 μm when tested according to USP <429> Laser Light Diffraction; and
b) the first SGM quantity if characterized by a d50 of from 98 to 516 μm when tested according to USP <429> Laser Light Diffraction.

33) The plurality of tablets of claim 26, wherein:
a) the tablets satisfy first and second dissolution specifications;
b) the first specification requires from 26 to 46% percent of metaxalone in the prototypes be released at 30 minutes under dissolution testing described in USP, Metaxalone Tablets; and
c) the second specification requires that at least 80 percent of metaxalone in the prototypes be released at 90 minutes under dissolution testing described in USP, Metaxalone Tablets.

34) The plurality of tablets of claim 26, packaged in a plurality of bottles, wherein the tablets from the first batch and the second batch are packaged in separate bottles.

35-51) (canceled)

47) An immediate release metaxalone tablet of metaxalone and one or more pharmaceutically acceptable tableting excipients that maintains a stable dissolution profile over a period of 6 months or more, when tested at 90 minutes under the dissolution testing described in USP, Metaxalone Tablets, comprising from 500 to 750, from 500 to 680, or 640 mg of metaxalone.

48) The tablet of claim 47, wherein the percentage of metaxalone released after 6 month differs by no more than 5%, 3%, or 2%, on an absolute basis, from the percentage of metaxalone released after 0 months or 3 months, when stored at accelerated condition of 40° C. and 75% relative humidity.

49) The tablet of claim 47, wherein the hardness of the tablet is balanced with the particle size of the metaxalone to produce the stable dissolution profile.

50) (canceled)

51) (canceled)
